(19) United States
(12) Patent Application Publication
(10) Pub. No.: US 2002/0054914 A1
(43) Pub. Date: May 9, 2002

(54) Compositions and methods for therapuetic agents complexed with calcium phosphate and encased by casein

(76) Inventors: Tulin Morcol, Decatur, GA (US); Steve J.D. Bell, Marietta, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/932,503

(22) Filed: Aug. 17, 2001

Related U.S. Application Data
(63) Continuation-in-part of application No. 09/496,771, filed on Feb. 3, 2000.
(60) Provisional application No. 60/118,356, filed on Feb. 3, 1999.
(60) Provisional application No. 60/118,364, filed on Feb. 3, 1999.
(60) Provisional application No. 60/118,355, filed on Feb. 3, 1999.
(60) Provisional application No. 60/267,357, filed on Feb. 9, 2001.

(51) Int. Cl. A61K 9/16; A61K 9/50; A61K 38/48; A61K 39/395; A61K 48/00; A61K 31/715

(52) U.S. Cl. 424/491; 424/493; 424/94.63; 424/130.1; 514/44; 514/171; 514/54

(57) ABSTRACT

The present invention relates generally to an oral drug delivery system which incorporates a therapeutic bioactive agent with biodegradable calcium phosphate particles, the particles then encapsulated by casein. The resulting particles provide a carrier designed to protect the therapeutic agent in the harsh, acidic environment of the stomach before releasing the agent into the small intestine. The therapeutic agent may be any therapeutically effective agent, such as a natural isolate or synthetic chemical or biological agent, such as a therapeutic agent, and in particular, may be a protein or a peptide such as insulin. Also incorporated with the particles may be additional surface modifying agents to assist binding, controlled release, or to otherwise modify the particles. The particles generally support the therapeutic agent to form controlled- or sustained-release particles for the oral or mucosal delivery of the therapeutic agent over time, wherein the therapeutic agent is incorporated into the structure of the particle core, disposed on the surface of the particle, or both.

This application is a continuation-in-part of U.S. patent application Ser. No. 09/496,771 filed on Feb. 3, 2000, which claims benefit of the filing dates of U.S. Provisional Application Ser. Nos. 60/118,356; 60/118,364; and 60/118,355, all filed Feb. 3, 1999, the entire contents of each of which are hereby incorporated by reference. This application also claims priority to U.S. Provisional Application No. 60/267,357 filed on Feb. 8, 2001, entitled “Casein-Complexation of Calcium Phosphate Particles Containing Insulin as Oral Delivery System,” the entire contents of which are hereby incorporated by reference.

### BACKGROUND OF THE INVENTION

1. Field of the Invention

This invention relates generally to calcium phosphate complexed with a therapeutic agent and at least partially encased or enclosed by casein micelles, to methods of making such particles, and to the oral delivery of therapeutic agents using such particles.

2. Description of Related Art

Treatment of many diseases, such as diabetes mellitus, usually requires daily subcutaneous injections of drugs, such as insulin. This can result in non-compliance of the patient because of the discomfort and inconvenience caused by multiple administrations. The oral delivery of such drugs would provide a more advantageous route of administration and may encourage patient compliance. However, oral administration of protein and/or peptide drugs, such as insulin, has traditionally been precluded by acid digestion of the drugs in the stomach and digestion in the small intestine. This is particularly true with proteins and peptides, which are difficult or impossible to administer orally since they are easily digested or hydrolyzed by the enzymes and other components of gastric juices and other fluids secreted by the digestive tract. Injection is often the primary alternative administration method, but is unpleasant, expensive, and is not well tolerated by patients requiring treatment for chronic illnesses. In particular, patients who are administered drugs on an cut-patient basis, or who self-administer, are more likely to fail to comply with the required administration schedule. A particular group of patients of this type are those suffering from diabetes, who frequently must inject themselves with insulin in order to maintain appropriate blood glucose levels.

Other drugs, compounds, or therapeutic agents that are desirable to be administered orally include, but are not limited to: Alpha-1-Antitrypsin; Human Growth Hormone (HGH); Erythropoeitin (EPO); Steroids, drugs to treat osteoporosis, blood coagulation factors, anti-cancer drugs, antibiotics, lipase, garanulocyte-colony stimulating factor (G-CSF), Beta-Blockers, anti-asthma, anti-sense oligonucleotides, therapeutic antibodies, DNase enzyme for respiratory and other diseases, anti-inflammatory drugs, anti-virals, anti-hypertensives, cardiotherapeutics such as anti-arrythmia drugs, and gene therapies, diuretics, anti-clotting chemicals such as heparin, combinations thereof, and any other agents adapted to be delivered orally.

For example, diabetes mellitus is a metabolic disease in which there is a deficiency or absence of insulin secretion by the pancreas. It is characterized by hyperglycemia, glycosuria, and alterations of protein and fat metabolism, producing polyuria, polydipsia, weight loss, ketosis, acidosis, and coma. See GOULD'S MEDICAL DICTIONARY, 381 4^{th} ed. 1979. Diabetes mellitus is often inherited, but it may be acquired. The disease occurs in two major forms: Type I, or insulin-dependent diabetes mellitus, and Type II, non-insulin-dependent diabetes mellitus. The condition may also be gestational (Type III), or due to impaired glucose tolerance (Type V). Type IV encompasses all other forms of diabetes, including those that are associated with pancreatic disease, hormonal changes, adverse effects of drugs, or genetic or other anomalies.

See www.harcourt.com/dictionary/def/2/9/4/9/2949900.html.

Specifically, Diabetes, Type I is an insulin-dependent diabetes (IDDM), now known to be a T-cell mediated autoimmune disease that specifically targets the pancreatic β-cells. It causes a deficiency strongly correlated to a hereditary predisposition to injury or destruction of pancreatic β-cells, which produce and secrete insulin. The β-cell insufficiency and destruction is generally caused by chemical-pH imbalances and viral or antibody damage, such as that caused by inflammatory cytokines, particularly those produced by TH1-type lymphocytes, which are hypothesized to play a major role in the pathogenesis of all autoimmune diseases, including diabetes of this type. Individuals are susceptible to Type I at an early age and usually suffer childhood onset. See http://vaxa.com/html/669.cfm.

Diabetes, Type II is a non-insulin dependent diabetes (NIDDM), being a disorder of glucose homeostasis characterized by hyperglycemia, peripheral insulin resistance, impaired hepatic glucose metabolism, and diminished glucose-dependent secretion of insulin from pancreatic β-cells. This latter defect may lie in the glucose signaling pathway in β-cells involving metabolically regulated potassium channels, which are the targets of sulphonylurea drugs commonly used in the treatment of NIDDM. Type II is characterized by insulin insensitivity, which is typically evidenced by high levels of circulating insulin and the reversibility of blood sugar elevation (by dietary changes and/or weight loss), sufficient to restore insulin sensitivity. Low GTF chromium levels are a major determinant of insulin insensitivity; obesity is another significant factor. Onset of Type II is generally diet related and usually occurs later in life. See id.

Treatment of diabetes mellitus usually requires daily subcutaneous injections of insulin. Because of the multiple administrations required, delivering insulin orally would provide a more advantageous route of administration, but the oral administration of insulin has traditionally been precluded by proteolytic degradation of the insulin in the stomach and upper portion of the small intestine.

Other drugs, compounds, or therapeutic agents that are desirable to be administered orally include, but are not limited to those described above.

More particularly, two general problems exist in developing oral insulin delivery systems (or any other protein or peptide drug oral delivery system). The major problem is the inactivation of insulin by digestive enzymes in the gastrointestinal system, mainly in the stomach and the proximal regions of the small intestine. One way researchers have attempted to overcome this problem is to prepare carriers that protect the insulin from the harsh environment of the stomach before releasing the drug into the more favorable regions of the gastrointestinal tract, specifically the colon. Insulin is susceptible to breakdown by proteases in the luminal cavity and the cells lining the mucosa. In attempts to combat this breakdown, researchers have incorporated protease inhibitors into various insulin formulations, which protects insulin degradation by the proteolytic enzymes. Other researchers have attempted to protect oral insulin from proteolytic degradation by including it within liposomes. However, the stability and effectiveness of insulin-contai ring liposomes has been found to be unpredictable.

Another major barrier to oral delivery of insulin is the slow transport of insulin across the lining of the colon into the bloodstream. In efforts to overcome this barrier, researchers have added absorption enhancers, which help facilitate the transport of macromolecules across the lining of the gastrointestinal tract. The resistance of the mucosal membrane to insulin penetration (in part because of insulin's large molecular size) is a factor limiting insulin diffusion across the biological membranes. Some researchers have studied the permeability of the small intestine to substances of high molecular weight and have found that the intestinal permeability is inversely proportional to molecular weight of the substance. The permeability of macromolecules has also been studied by using surfactants. Cyclodexrins have also been used in an attempt to enhance enteral absorption of insulin in the lower jejunal/upper ileal segments of rats. However, the enhancer approaches are often unsuccessful because the enhancers have little selectivity regarding the actions of the permeants. Accordingly, some researchers believe that prolonging the residence time in the absorption site would be effective in enhancing the absorption of poorly permeable drugs—if they can be protected from the degradation.

In general, the need to find a system for oral administration of insulin has resulted in many investigations and studies focused on protecting the molecule from degradation and facilitating the transport of the intact molecule. Researchers have formulated and studied a variety of delivery mechanisms and methods in order to provide a carrier system for oral delivery of insulin. Various approaches, such as alternative routes, absorption enhancers, protease inhibitors, chemical modification, and dosage forms, have been examined to overcome the delivery problems of peptides and proteins via the gastrointestinal tract.

For example, researchers have attempted to deliver insulin to the more distal portions of the gastrointestinal tract by microencapsulation using Eudragit RS 100 or encapsulation by liposomes. Researchers have also attempted to use polymeric structures formed by polymerization of isobutyl cyanoacrylate in an acidic medium to encapsulate insulin. One limitation of these formulations is that it is difficult to remove organic solvents from the final product. These procedures also present the possibility of undesired structural modification of the drug.

Additional efforts to use polymeric carriers as oral delivery systems have included encapsulating insulin within polyacrylates, as well as dispersing insulin in a terpolymer of styrene and hydroxyethyl methacrylate cross-linked with a difunctional azo-containing compound. In these studies, the polymer degrades, allowing for controlled release of the insulin into the colon. In addition, researchers have used hydrogel systems that contain immobilized insulin and protease inhibitors; have coated insulin with an impermeable film, which is cleaved in the colon by the microflora, thus releasing insulin; have added insulin to a polymeric drug carrier composed of polyalkylcyanoacrylates; have bound insulin to erythrocyte membranes for oral administration; have prepared capsules using chitosan (a high molecular weight cationic polysaccharide derived from naturally occurring chitin in crab and shrimp shells by deacetylation); have incorporated insulin into a gel-like material made primarily of a combination of polymers, such as polymethacrylic acid and polyethylene glycol; and have developed insulin-containing poly(anhydride) microspheres.

These efforts have generally been directed to finding materials that adhere to the walls of the small intestine and release insulin based on degradation of the polymer carrier. For a general discussion of the efforts described above, see generally, A. M. Lowman, Oral Delivery of Insulin Using pH-Responsive Complexation Gels, 88 JOURNAL OF PHARMACEUTICAL SCIENCES, 933 (1999); C. T. Musbayne, et al., Orally administered, insulin-loaded amidated pectin hydogel beads sustain plasma concentrations of insulin in streptozotocin-diabetic rats, 164 JOURNAL OF ENDOCRIMOLOGY, 1 (2000); E. A. Hosney, Hypoglycemic Effect Of Oral Insulin in Diabetic Rabbits Using pH-Dependent Coated Capsules Containing Sodium Salicylate Without And With Sodium Cholate, 24(3) DRUG DEVELOPMENT AND INDUSTRIAL PHARMACY 307, 308 (1998).

Additionally, the colon, the region of gastrointestinal tract with the lowest peptidase activity, has also been investigated as an attractive absorption site for orally administered protein drugs. Pectin has been investigated for specific delivery to the colon because it can form insoluble hydrophilic matrices which are not degraded by gastric or intestinal enzymes, but degraded by pectinolytic enzymes of the colon. See C. T. Musbayne, et al., Orally administered, insulin-loaded amidated pectin hydogel beads sustain plasma concentrations of insulin in streptozotocin-diabetic rats, 164 JOURNAL OF ENDOCRINOLOGY, 1 (2000). The researchers studied the oral administration of insulin entrapped in amidated pectin hydrogel beads and found that the pectin-hydrogel beads administered in a single dose of 46 micrograms of insulin was more effective than two doses delivering 30 micrograms given about eight hours apart. The researchers hypothesized that these observations could be attributable to the transit time of individual beads, enzymatic breakdown of the beads, and the influence of food.

The hypoglycemic effect of Eudragit RS 100 coated capsules containing insulin and sodium salicylate when given orally has also been compared with insulin suspensions given subcutaneously. See E. A. Hosney, Hypoglycemic Effect Of Oral Insulin in Diabetic Rabbits Using pH-Dependent Coated Capsules Containing Sodium Salicylate Without And With Sodium Cholate, 24(3) DRUG DEVELOPMENT AND INDUSTRIAL PHARMACY 307, 308 (1998). The researchers found that salicylates promoted absorption of the insulin. Specifically, when insulin was administered orally in a pH-dependent Eudragit coated capsule forn with sodium salicylate, a significant reduction in plasma glucose level was found. The maximum reduction reached was 56% of initial values, whereas average levels reached with subcutaneous administration of insulin reached 34-35% of initial values. Additionally, capsules that did not contain salicylate or that were not coated with Eudragit did not produce any reduction in plasma glucose levels.

Other researchers have studied liposomes as carriers for oral administration of enzyme, focusing on the fact that there has been little success in achieving acceptable bioavailability of insulin when it is delivered orally due to extensive inactivation of the insulin by gastrointestinal enzymes. See K. D. Choudhari, et al., Liposomes As Carrier for Oral Administration of Insulin: Effect of Formulation Factors, 11 (3) JOURNAL OF MICROENCAPSULATION, 319 (1994). Generally, the use of liposomes as a carrier for drugs depends upon various factors, such as composition of the liposome membrane, encapsulating efficiency, stability, release rates, body distribution after administration, liposorne size, surface charge, size distribution, and the type of drug used. Researchers have found that liposome encapsulated insulin was comparable to the effect of insulin given subcutaneously. See id. at 324.

Researchers have also studied poly(vinyl alcohol) gel spheres as an oral drug delivery system. See T. Kimura, et al., Oral Administration of Insulin as Poly(Vinyl Alcohol)-Gel Spheres in Diabetic Rats, 19(6) BIOL.PHARM. BULLETIN, 897 (1996). The gel spheres provided a prolonged residence in the small intestine, which is the major site of drug absorption. The researchers found that the gel spheres enabled prolonged residence time in the ileum, but that the release of insulin and the protease inhibitor from the gel spheres should be synchronized to insure the protease inhibitor's anti-proteolytic effect. See id. at 899. The insulin and protease inhibitors showed a similar release from the gel spheres, suggesting that diffusion in the gel matrix is the rate-determining step for pepti de release. The gel spheres released insulin and the protease inhibitor slowly, resulting in an incomplete protective effect in the jejunum with high degrading activity. On the other hand, in spite of their slow releasing property, the gel spheres were effective in the lower intestine where the proteases were less active. See id. at 890.

Another attempt to deliver insulin orally is described in U.S. Pat. No. 5,843,887, titled “Compositions for Delivery of Polypeptides, and Methods,” issued to Petit et al. This patent discloses an insulin/Intrinsic Factor combination that can be delivered orally. (Intrinsic Factor is a glycoprotein with a molecular weight of 50 kDa and comprises 351 amino acids and 15% carbohydrate.) The Intrinsic Factor protects the insulin from the action of proteolytic enzymes in the gastrointestinal tract. When administered orally, the insulin/Intrinsic Factor combination produces a fall in serum glucose, whereas no change in serum glucose was noted when insulin alone was administered orally. Intrinsic Factor is placed in a buffered medium and a polypeptide of interest (such as insulin) is added to the solution so that the intrinsic factor acts as a carrier for the polypeptide while protecting the polypeptide and facilitating its release.

U.S. Pat. No. 6,017,545 issued to Modi is directed to delivery of macromolecular pharmaceutical agents, particularly insulin, through membranes in the nose and mouth. A protein drug is encapsulated in mixed micelles and applied to mucosal membranes. The mixed micelles are smaller than the pores of the membranes in the oral cavity or the gastrointestinal tract to help the encapsulated molecules penetrate efficiently through mucosal membranes. The insulin-containing compounds may also contain at least one inorganic salt, such as sodium, potassium, calcium and zinc salts. The inorganic salts help open the channels in the gastrointestinal tract and may provide additional stimulation to release the insulin.

U.S. Pat. No. 5,428,066 to Lamer et al. is directed to a method of treating elevated blood sugar by administering an insulin mediator containing chiro-inositol. The chiro-inositol may be administered alone or together with additives. It may be administered as a tablet containing chiro-inositol combined with excipients, for example, inert diluents such as calcium carbonate, sodium carbonate, lactose, calcium phosphate, or sodium phosphate. The tablets may be uncoated or coated to provide sustained action. Time release materials, such as glyceryl monostearate or glyceryl distearate, alone or with a wax may be employed. The active ingredient may also be presented as a gelatin capsule.

Generally, this reference focuses on treating insulin-resistance by administration of an insulin mediator, rather than on administering insulin per se. The tablets and gelatin capsules produced using conventional coating agents (e.g. wax, glyceryl monostearate) and inert diluents (e.g. calcium carbonate, calcium phosphate) can delay disintegration and adsorption of small, “non-protein drugs,” such as chiro-inositol, in the gastrointestinal (GI) tract. However, they alone cannot prevent proteolytic degradation of protein or peptide drugs in the gastrointestinal tract.

U.S. Pat. No. 5,665,382 to Grinstaff et al. titled “Methods for the Preparation of Pharmaceutically Active Agents for In Vivo Delivery,” discloses compositions used to deliver a biologic contained within a polymeric shell. The polymeric shell is a biocompatible material, crosslinked by the presence of disulfide bonds. It is formed of biocompatible materials such as proteins, polypeptides, oligopeptides, polynucleotides, polysaccharides, starch, cellulose, as well as synthetic polypeptides. In some embodiments, the biologic material may form part of the polymeric shell itself. The polymeric shell may also have a small amount of PEG-containing sulfhydryl groups included with the polymer. A critical feature is that the polymeric shell is crosslinked through the formation of disulfide bonds.

U.S. Pat. No. 5,110,606 to Geyer et al. is directed to a palatable liquid therapeutic emulsion used for drug delivery. A drug, such as ibuprofen, aspirin or a vitamin, is dissolved in a solvent, such as glycerin, polypropylene glycol or polyethylene glycol. The drug can be supersaturated without crystallizing.

None of the references described herein suggest or disclose the use of a calcium phosphate/insulin core with casein micelles reconstructed as aggregates around the cores, forming micellar structures. More particularly, none of the references disclose or suggest complexing a therapeutic agent, for example, insulin, with calcium phosphate, and then encasing at least a portion of the complexed calcium phosphate/therapeutic agent particle with casein. Although some of the references describe the oral delivery of insulin using various gels, liposomes, and lipid emulsions, none specifically consider or disclose using calcium phosphate or casein micelles as delivery mechanisms for the insulin.

Nanometer scale particles have been proposed for use as carrier particles, as supports for biologically active molecules, such as proteins, and as decoy viruses. See U.S. Pat. Nos. 5,178,882; 5,219,577; 5,306,508; 5,334,394; 5,460,830; 5,460,831; 5,462,750; and 5,464,634, the entire contents of each of which are hereby incorporated by reference. The particles disclosed in the above-referenced patents, are generally extremely small, in the 10-200 nm size range.

One reference discussing calcium phosphate particles is Application WO 00/15194, published Mar. 23, 2000, issued to Lee and assigned to Etex Corp., using calcium phosphate as an adjuvant and delivery vehicle for active agents such as antigens, vaccines, second adjuvants, bacteria, viruses, or fragments thereof, nucleic acids, proteins, heat shock proteins (HSP's) haptens, tolergens, allergens, immunogens, antibiotics or other active moieties. The calcium compound is formed into an injectable gel or solid nanoparticles and is delivered by injection, by transdermal and/or mucosal delivery, as a suppository, as an inhalant, spread as a paste, or implanted surgically.

With respect to casein, some references have suggested the benefits of protective hydrolyzed casein(HC)-based diets to decrease diabetes frequency and the severity of insulitis. See Elizabeth Olivares, et al., Effects of a Protective Hydrolized Casein Diet Upon the Metabolic and Secretory Responses of Pancreatic Islets to IL-1β, Cytokine Production by Mesenteric Lymph Node Cells, Mitogenic and Biosynthetic Activities and Peyers' Patch Cells, and Mitogenic Activity and Pancreatic Lymph Node Cells from Control and Diabetes-Prone BB Rats, 68 MOLECULAR GENETICS AND METABOLISM 379, 380 (1999), For example, a website that markets formulas to regulate proper glucose metabolism states: “a diet rich in Casein appears to actually protect subjects (non-obese mice who have a genetic predisposition for developing diabetes: NOD mice) from developing diabetes and then passing it on to their young. Specifically, Casein fed NOD female mice were protected against spontaneous diabetes and male NOD mice against acute Cyclosphosphamide or Cy-induced diabetes while also lessening the severity of insulitis.” See vaxa.com/html/669.cfm.

Researchers have also studied the benefits of using casein as a delivery system for various drugs. For example, researchers have studied casein microspheres as a carrier system for doxorubicin. The carriers were prepared by mixing casein with a doxorubicin solution and adding lactose as an excipient. In one embodiment, the drug was incorporated as a complex with polyaspartic acid. See Yan Chen, et al., Comparison of albumin and casein microspheres as a carrier for doxorubicin, 39 J. PHARM. PHARMACOL., 978-85 (1987). The researchers found that doxorubicin drug release rates from casein microspheres were slower than from the albumin systems, even though there was less drug in the casein microsphere.

Controlled release of theophylline using casein as the matrix has also been studied. See M. S. Latha, Glutaraldehyde cross-linked bovine casein microspheres as a matrix for the controlled release oftheophylline: in0vitro studies, 46(1) J. PHARM. PHARMACOL, 8-13 (1994). The researchers describe forming drug-loaded microspheres by glutaraldehyde cross-linking of an aqueous alkaline solution of casein containing the drug dispersed in a mixture of dichloromethane/hexane with an aliphatic polyurethane as the suspension stabilizer. The same researchers have also studied the casein microspheres loaded with 5-fluorouracil. See M. S. Latha, et al., Casein as a carrier matrix for 5-fluorouracil: drug release from microspheres, drug-protein conjugates and in-vivo degradation microspheres in rat muscle, 46(11) J. PHARM. PHARMACOL, 858-62 (1994).

Casein microspheres have also been loaded with mitoxantrone for use as, a drug delivery mechanism. See W. A. Knepp, Synthesis, properties, and intratumoral evaluation of mitroxantrone-loaded casein microspheres in Lewis lung cacimnoma, 45(10) J. PHARM. PHARMACOL, 887-91(1993). The article discusses post-synthesis loading of mitoxanthrone onto casein microspheres containing 20% polyglutamic acid and relates to intratumoral administration of the particles, not oral administration.

Another article studying the effects of casein microparticles as a delivery system has shown that lactic acid plus hydoxypropyl methycellulose and gelatin results in a biodegradable and homogeneous casein microparticle, presenting a potentially useful drug delivery system. See Ana J. P. Santinho, et al., Influence of formulation on the physiochemical properties of casein microparticles, 186 INTL. JOURNAL OF PHARMACEUTICS, 191-98 (1999). Other articles report using casein to deliver 5-fluorouracil and progesterone. See N. Willmott, et al., Doxorubicin-loaded casein microspheres: protein nature of drug incorporation, 44(6) J. PHARM. PHARACOL, 472-75 (1992); M. S. Latha, Progesterone release from glutaraldehyde cross-linked casein microspheres: in vitro studies and in vivo response in rabbits, 61(5) CONTRACEPTION, 329-34 (2000).

Despite the above-described attempts, there remains a need for an oral delivery system that effectively provides consistent, reliable, therapeutic blood levels of therapeutic agents, and in particular, of insulin and other hormones. It is particularly desirable that the delivery system be able to withstand proteolysis, to prevent degradation of the therapeutic agents before it can be delivered. Therefore, there is a need for calcium phosphate particle cores that are useful as core materials or carriers for biologically active moieties which can be produced simply and consistently, that can deliver a therapeutic agent, and that can be protected for oral administration of such agent.

### SUMMARY OF INVENTION

The present invention relates generally to an oral drug delivery system which incorporates a therapeutic bioactive agent with biodegradable calcium phosphate (CAP) particles, which particles are dispersed in an aqueous solution or dispersion of caseins to re-precipitae caseins (reform casein micelles) and as a result, drug-loaded particles are encapsulated by a protective layer comprising complexed caseins and/or casein micelles. For purposes of this document, “encapsulated” “embedded” or “incorporated” means complexed, encased, bonded with, related to, at least partially coated with, layered with, or enclosed by a substance. The resulting complex provide a carrier designed to protect the therapeutic agent in the harsh, acidic environment of the stomach before releasing therapeutic agent into the small intestine. The therapeutic agent may be any therapeutically effective agent, such as a protein, a peptide, a hormone, such as insulin, and even more particularly, recombinant or native human insulin, a steroid, an enzyme, a small drug molecule, a therapeutic antibody, a vaccine antigen, any of the agents described above, or any combination thereof.

Also incorporated with the particles may be additional surface modifying agents to assist binding, controlled release, or to otherwise modify the particles. In other words, the particles may be coated or complexed with an additional surface modifying agent or they may remain uncoated. In either embodiment, the particles support a therapeutic agent to form controlled release particles for the sustained release of the therapeutic agent over time, wherein the therapeutic agent is incorporated into the structure of the particle core, disposed on the surface of the core, or both.

More particularly, incorporating the additional surface modifying agent and/or the therapeutic agent into the CAP particles may be carried out during particle synthesis (“inside formulation”) or the surface modifying agent and/or the therapeutic agent may be at least partially coated on the outside of the CAP particles once they have been formed (“outside formulation”) or both (called the “inside/outside” formulation). The final particles are then complexed with either commercially available processed casein or otherwise prepared casein to re-construct casein micelles around the CAP-therapeutic agent-optional surface modifying agent core.

The present invention provides a particle comprising a core, comprising calcium phosphate, a therapeutic agent associated with the core, and a protective lipophilic coat comprising casein and/or reformed casein micelles at least partially covering the core. In a more particular embodiment, the invention provides a therapeutic composition suitable for oral or mucosal delivery of insulin, comprising a core comprising calcium phosphate, insulin and polyethylene glycol associated with the core, wherein the insulin and polyethylene glycol are at least partially embedded in the core, and a protective layer comprising casein and/or reformed casein micelles at least partially covering the core. The casein- encapsulated particles of insulin can be combined with a pharmaceutically acceptable excipient or can be dried and specific doses can be dispensed in any conventional oral drug delivery system, such as hard or soft gelatin capsules.

The invention also provides a method for preparing one or more particles comprising reacting a soluble calcium salt, a soluble phosphate salt, a soluble citrate salt, and the therapeutic agent to form a mixture and dispersing the mixture in an aqueous dispersion of casein. Furthermore, the invention relates to a method for orally delivering therapeutic amounts of insulin as an oral dosage form to a patient in need thereof.

Thus, the present invention relates to compositions for the oral delivery of therapeutic agents, to methods of preparing such compositions, and to methods of using these compositions as controlled release matrices for the oral delivery of therapeutic agents. The present invention also relates to methods of increasing bioavailability of therapeutic agents and treating medical conditions that benefit from administration of therapeutic agents by administering effective amounts of the particles of this invention to a patient in need thereof via oral delivery.

### BRIEF DESCRIPTION OF THE FIGURES

FIG. 1 is a schematic drawing showing a calcium phosphate particle core (4) both coated with therapeutic agent (8) and having therapeutic agent (8) impregnated therein.

FIG. 2 is a series of schematic drawings showing various embodiments of the calcium phosphate core of the oral composition of this invention. FIG. 2A shows a particle coated directly with therapeutic agent (6). FIG. 2B shows a particle (4) coated with surface modifying agent (2), such as polyethylene glycol or monosaccharide or disaccharide sugar such as cellobiose, and a having a therapeutic agent (6) adhered to the surface modifying agent (2). FIG. 2C shows a particle (4) having a surface modifying agent (2). such as polyethylene glycol or monosaccharide or disaccharide sugar such as cellobiose incorporated therein and having a therapeutic agent (6) at least partially coating the particle (4).

FIG. 3 is a schematic drawing showing the particle core of the present oral drug composition (4) having both a surface modifying agent (2), such as polyethylene glycol or monosaccharide or disaccharide sugar such as cellobiose and a therapeutic agent (6) incorporated therein.

FIG. 4 is a bar graph showing the results and stability of a formulation of the present invention against digestive enzyme pepsin in pH 1.5 and pH 3 glycine buffer. Forty IU/ml insulin either free in solution, in CAPI formulation, or CAPIC- 1 formulation was incubated in 10 IU/ml pepsin for 30 minutes at 37° C.

FIG. 5 is a graph showing the blood glucose levels in fasted diabetic mice after graded doses of oral insulin in casein-coated particles of the present invention.

### DETAILED DESCRIPTION OF SPECIFIC EMBODIMENTS

There are three main elements that comprise the composition of the present invention: calcium phosphate (CAP), a therapeutic agent (TA), and casein (C). The calcium phosphate particles containing the therapeutic agent form the core of the present oral formulation. The particle cores may have a therapeutic agent coated thereon (“outside formulation”), embedded or impregnated therein (“inside formulation”), or a combination of both, i.e., a coating on the outside of the particle cores, as well as the therapeutic agents being dispersed within the particle cores (“inside/outside formulation”). The cores may optionally have an additional surface modifying agent coating the core, embedded or impregnated within the core, or a combination of both. In each embodiment, the particles (i.e., the core, the therapeutic agent, and any surface modifying agent) are then encased in or otherwise complexed with casein for oral delivery of the particles, such that the casein component of the composition surrounds and coats the particles and provides protection of the therapeutic agent against digestive enzymes of the stomach. The present invention also relates to a method of reconstructing casein micelles as aggregates around calcium phosphate particles complexed with a therapeutic agent to provide a protective coat surrounding the particles. The casein micelles are reformed around a therapeutic agent or an active “protein drug” (e.g. insulin) to mediate its passage through the acidic environment of the gastrointestinal tract. Once the protein-containing particles are coated by casein or casein micelles, conventional tablet and capsule manufacturing procedures for oral administration may also be used to further control the protein's adsorption by the gastrointestinal tract.

The casein protective coat around the particle cores will be in a collapsed conformation in acidic media, such as the gastric fluid and the acidic pH of gastrointestinal tract, due to agglomeration of micelles. The release of the therapeutic agent from the formulation will be triggered in less acidic media of pH greater than about 5.5, such as in the small intestine, where the collapsed conformation will begin to loosen (i.e., to relax or spread out), allowing the therapeutic agent to diffuse into the surrounding tissue and eventually into the blood stream.

There is reason to believe that at the acidic pH conditions of the stomach, the solubility of casein-coated composition comprising the therapeutic agent in its core will considerably decrease. Without wishing to be bound to any theory, it is believed that even though the calcium phosphate is solubilized, acidic pH will decrease the swelling of the casein complex containing the therapeutic drug, and digestive enzymes which degrade proteins will diffuse only minimally, if at al, through the close network of the cuter casein clusters. As the protected drug escapes from the harsh environment of the stomach into the intestines, the pH of the environment, as well as high peptidase activity in the small intestine, increases. Due to casein's natural adhesive properties, the drug-casein complex will tend to adhere to the membrane of the gastrointestinal tract. The collapsed structure of the casein complex will start swelling and relaxing into a more open structure. Casein biodegradation also increases, releasing physiologically effective amounts of the therapeutic drug through the walls of small intestines into the blood stream.

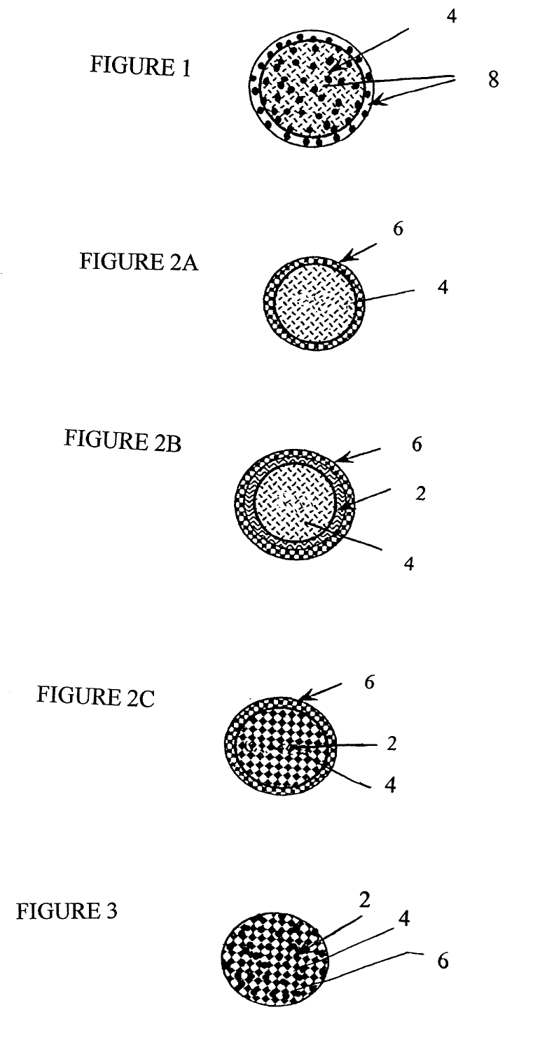

The casein molecules are arranged, presumably as micelles, around calcium phosphate particles containing the active drug, and are linked to the therapeutic agent-containing microparticles by mainly calcium phosphate and electrostatic bond interactions. Without wishing to be bound to any theory, it is believed that the composition simulates general properties of casein micelles; i.e., insoluble in water, very stable, can be dispersed in a non-aggregatory colloidal phase in natural pH or alkali buffers, and the surface of the therapeutic composition is highly hydrophilic and negatively charged. Again, without wishing to be bound by any theory, it is believed that the casein molecules form clusters “glued” by calcium phosphate, rather than forming a complete shell around the particle. In other words, it is believed that the calcium phosphate particles encapsulating the drug protein and caseins are held together by association and electrostatic charge interactions but not by covalent bonding. Thus, the casein-coated particles of the present invention may or may not be spherical in shape and will most likely not have a smooth surface, even though schematic FIGS. 1-3 show them as spherical for ease of illustration.

If the calcium phosphate particles are formulated initially and the therapeutic agent and/or surface modifying agent is coated thereon, the following procedure provides one specific embodiment for the preparation such particles.

Formation of Particles.

The calcium phosphate particle core of the present invention is typically prepared as a suspension in aqueous medium by reacting a soluble calcium salt with a soluble phosphate salt, and more particularly, by reacting calcium chloride with sodium phosphate under aseptic conditions. Initially, an aqueous solution of calcium chloride having a concentration between about 5 mM and about 250 mM is combined by mixing with an aqueous solution of a suitable distilled water-based solution of sodium citrate, having a concentration between about 5 mM and about 250 mM. It is believed that the presence of sodium citrate contributes to the formation of an electrostatic layer around the particle core, which helps to stabilize the attractive and repulsive forces between the particle cores, resulting in physically stable calcium phosphate particle cores.

An aqueous solution of dibasic sodium phosphate having a concentration between about 5 mM and about 250 mM is then mixed with the calcium chloride/sodium citrate solution. Turbidity generally forms immediately, indicating the formation of calcium phosphate particles. Mixing is generally continued for at least about 48 hours, or until stable particle formation has been obtained, as determined by sampling the suspension and measuring the particle size using known methods. The particles may be optionally produced in the nanometer size range (50-1000 nm) using a sonicator. The unloaded particles may be stored and allowed to equilibrate for about seven days at room temperature to achieve stability in size and pH prior to further use. Example 1 below provides an exemplary embodiment of one method that may be used to prepare particles for use in this invention.

Additional Surface Modifying Agent Coating.

In order to coat and adhere a therapeutic agent to the formed particle core, an optional surface modifying agent, may be used. For example, surface modifying agents suitable for use in the present invention include substances that provide a threshold surface energy to the particle core sufficient to bind material to the surface of the particle core, without denaturing the material. Example of suitable surface modifying agents include those described in U.S. Pat. Nos. 5,460,830, 5,462,751, 5,460,831, and 5,219,577, the entire contents of each of which are incorporated herein by reference. Non-limiting examples of suitable surface modifying agents may include basic or modified sugars, such as cellobiose, or oligonucleotides, which are all described in U.S. Pat. No. 5,219,577. Suitable surface modifying agents also include carbohydrates, carbohydrate derivatives, and other macromolecules with carbohydrate-like components characterized by the abundance of —OH side groups, as described, for example, in U.S. Pat. No. 5,460,830. Polyethylene glycol (PEG) is a particularly suitable surface modifying agent.

In this embodiment, the particle cores may be at least partially coated with the surface modifying agent by preparing a stock solution of a surface modifying agent, such as cellobiose (e.g., around 292 mM) or PEG (e.g. 10% w/v) and adding the stock solution to a suspension of calcium phosphate particle cores at a ratio of about 1 mL of stock solution to about 20 mL of particle suspension. The mixture can be swirled and allowed to stand overnight to form at least partially coated particle cores. The at least partially coated particle cores are adapted to have a therapeutic agent adsorbed thereon. Generally, this procedure will result in a substantially complete coating of the particles, although some partially coated or uncoated particles may be present.

Therapeutic Agent Coating.

A therapeutic agent is then attached or otherwise coated onto the particles. Desirably, this therapeutic agent will benefit from increased protein from the gastric environment. Therapeutic agents suitable for use with the present invention include, but are rot limited to insulin, Alpha-1-Antitrypsin, Human Growth Hormone (HGH), Erythropoeitin (EPO), Steroids, drugs to treat osteoporosis, blood coagulation factors, anti-cancer drugs, antibiotics, lipase, garanulocyte-colony stimulating factor (G-CSF), Beta-Blockers, anti-asthma, anti-sense oligonucleotides, therapeutic antibodies, DNase enzyme for respiratory and other diseases, anti-inflammatory drugs, anti-virals, anti-hypertensives, cardiotherapeutics such as anti-arrythmia drugs, and gene therapies, diuretics, anti-clotting chemicals such as heparin, combinations thereof, and any other agents adapted to be delivered orally. The agent may be either a natural isolate or synthetic, chemical or biological agent, and in particular, may be a protein or a peptide.

Coating of the particle cores with a therapeutic agent is preferably carried out by suspending the particle cores in a solution containing a surface modifying agent, generally a solution of double distilled water containing from about 0.1 to about 30 wt % of the surface modifying agent. The particles are maintained in the surface modifying agent solution for a suitable period of time, generally about one hour, and may be agitated, e.g., by rocking, stirring, or sonication, to form at least partially coated particles. Generally, this procedure will result in substantially complete coating of the particles, although some partially coated or uncoated particles may be present.

The at least partially coated particle cores can be separated from the suspension, including from any unbound surface modifying agent, (if used) by centrifugation. The at least partially coated particle cores can then be resuspended in a solution containing the therapeutic agent to be adhered to the at least partially coated particle core. Optionally, a second layer of surface modifying agent may also be applied to the therapeutic agent adhered to the particle. Further, a second layer of therapeutic agent may also be applied to the second layer of surface modifying agent, and so on.

In another embodiment, a therapeutic agent may be attached to an unmodified particle surface, although particles at least partially coated with a surface modifying agent generally have greater loading capacities. For example, insulin loading capacities of at least partially coated particles have been found to be about 3 to 4-fold higher than insulin loading capacities of unmodified particle surfaces. Particle cores coated or impregnated with a material (6), such as a therapeutic agent, preferably a protein or peptide, and more preferably human insulin, are shown in FIGS. 2 and 3.

Surface Modifying Agent Incorporated in Particle with Therapeutic Agent Coating.

Another embodiment that facilitates higher loading capacities is schematically illustrated in FIG. 2C, which shows a particle core having a surface modifying agent (2), such as polyethylene glycol, impregnated therein. The particles may be prepared by adding a surface modifying agent (2) to one or more of the aqueous solutions forming the particle core (4). The particle cores may optionally be stored at room temperature. To obtain at least partially coated particles, the particles are subsequently contacted with a therapeutic agent, such as a protein or peptide such as insulin, and more particularly human insulin, to provide at least a partial coating on the particle as described above.

Therapeutic Agent and Surface Modifying Agent Incorporated in Particle.

A further embodiment facilitating higher loading capacities is illustrated in FIG. 3, which shows a particle core (4) having both a surface modifying agent (2), such as polyethylene glycol, and a therapeutic agent (6), incorporated therein or co-precipitated. One way in which particles of this embodiment may be prepared is by combining a therapeutic agent, such as insulin and/or any other desired agent and an optional surface modifying agent together to form a solution. This solution is then combined with one or more of the aqueous solutions forming the particle as described above. The resulting particles incorporate calcium phosphate, surface modifying agent, and therapeutic agent within the particle structure. Example 3 below provides an exemplary embodiment of one method that may be used to prepare particles having a therapeutic agent and a surface modifying agent embedded therein.

Particles prepared according to this and any other embodiments described herein may be combined with one or more particles prepared according to any other embodiment described herein. Moreover, as described, the particles described above may be formed without the surface modifying agent. That is, the particles may comprise only calcium phosphate and a therapeutic agent. Particles according to this embodiment are formed as described above, without the surface modifying agent being added to solution, i.e., by directly adding the therapeutic agent with the reactants forming the calcium phosphate particles being formed or by adding the therapeutic agent to solution once the particles have already formed.

Incorporating a therapeutic agent into the particle may be accomplished by mixing an aqueous calcium chloride solution with the therapeutic agent to be incorporated prior to combining and mixing with either the sodium citrate or dibasic sodium phosphate solutions, to co-crystallize the calcium phosphate particle cores with the therapeutic agent.

Protective Casein Coating.

The composition described above, comprising calcium phosphate complexed with a therapeutic agent and/or a surface modifying agent at least partially coating or impregnating or both the calcium phosphate is then encased, enclosed by, or otherwise complexed with casein. This forms an oral delivery system adapted to protect the therapeutic agent from proteolytic degradation in the gastrointestinal tract and to be administered to patients in need of the therapeutic agent. Preferably, the casein micelles are reconstructed around the particles.

The particles as formed above are suspended in a casein dispersion and gently stirred. Reformed casein micelles containing the CAP-therapeutic agent may be collected by centrifugation and lyophilized to dryness. In another embodiment, a re-formed casein micelle suspension enclosing the therapeutic material may be sonicated to break up possible clump formations due to casein-casein interactions (adhesions), and then may be lyophilized. Sonication time may be adjusted to tailor the average casein-coated subunit sizes for other routes of drug delivery, including but not limited to pulmonary, intra muscular, or subcutaneous injections. Examples 4 and 6 below provide examples of methods that may be used to prepare particles having a casein coating according to various embodiments of this invention.

Generally, casein micelles retain their integrity in aqueous mixtures of pH between about 6.3 to about 7 and agglomerate in acidic mediums of pH lower than about 5. Commercial casein products are commonly prepared by reducing the pH of milk to pH of about 4.6, thus destroying the micelle structures and precipitating caseins in solid form. One specific method of reforming casein micelles comprises removing the micellar calcium phosphate from milk by contacting the milk with a chelating agent, such as EDTA and sodium citrate, to disrupt the casein micelles, and then introducing divalent cationic salts, such as calcium phosphate, to reconstitute the micelles. The micelles are re-constructed around insoluble calcium phosphate salts, and for the purposes of the present invention, preferably around calcium phosphate particles. This method is described by U.S. Pat. No. 6,183,803, titled “Method for Processing Milk,” hereby incorporated herein by this reference.

In that patent, the inventors provided a method of deconstructing the micelles (using a metal chelating agent) and re-constructing them again around insoluble divalert cationic salts, particularly calcium phosphate particles. The present invention relates to a method of reconstructing casein micelles around therapeutic agent-loaded CAP particles for the purpose of creating a protective coat surrounding the CAP-therapeutic agent particles, which will be in a collapsed conformation in acidic media, such as the gastric fluid of the stomach, due to agglomeration of micelles. The release of therapeutic agent from the formulation will be in less acidic media of pH greater than 5.5, such as in the small intestine, where the collapsed conformation will start to relax, allowing the drug to diffuse into the surrounding tissue and eventually into the blood stream.

Reformed casein micelles comprise an aggregate of caseins linked together with insoluble calcium phosphate clusters or particles. The size of the reformed casein micelle primarily depends upon the size of the insoluble calcium phosphate particles and micelle-micelle interactions. The calcium phosphate particles of the present invention may be nanoparticles, as described in U.S. Pat. No. 5,462,751 and in Patent Application Ser. No. 09/496,771, hereby incorporated herein by reference or they may be microparticles. In a particular embodiment, the calcium phosphate particles of the present invention range from about 300-4500 nm and preferably, 300-3000 nm. Alternatively, the particles may comprise clusters of larger units (larger than 4500 nm) but can be sonicated to have smaller subunits if needed.

In a further embodiment, the particles of the present invention may be microparticles ranging from about 1 μm to 10 μm, and the reformed casein micelles are in the micrometer size range. In addition, the particles of the present invention may be combined with a pharmaceutically acceptable excipient or encapsulated in conventional oral delivery systems for delivery.

The biological activity of the therapeutic agent is substantially preserved using the present method. While not wishing to be bound to any theory, it is believed that the casein micelle surface, mostly provided by k-caseins, forms a hydrophilic “hairy” layer that facilitates steric and electrostatic repulsive forces around the encapsulated protein-drug.

In order to test the CAP-therapeutic agent-casein formulation against digestive enzymes and thus, demonstrate its usefulness as an oral delivery system, Example 5 provides a CAP-therapeutic agent-casein formulation mixed with pepsin or other digestive enzyme found in the gastric juices that catalyze the breakdown of protein to small peptides and amino acid units.

The various embodiments of the invention can be more clearly understood by reference to the following nonlimiting examples.

### EXAMPLE 1

CAP Particles.

A 12.5 mM solution of CaCl₂ is prepared by mixing 1.8378 g of CaCl₂ into 800 mL of sterile GDP water under aseptic conditions until completely dissolved, and the solution diluted to 1 L and filtered. A 15.625 mM solution of sodium citrate was prepared by dissolving 0.919 g of sodium citrate into 200 mL of sterile GDP water with mixing using aseptic techniques and filtered. A 12.5 mM solution of dibasic sodium phosphate was prepared by dissolving 1.775 g sodium phosphate into 1 L of sterile GDP water with mixing using aseptic techniques and filtered. All solutions were stored at room temperature.

The calcium chloride solution was combined with the sodium citrate solution and thoroughly mixed. Subsequently, the sodium phosphate solution was added with mixing. Turbidity appeared immediately as particles began to form. The suspension was allowed to mix for several minutes and was sampled for endotoxin testing using aseptic technique. Mixing was continued for about 48 hours under a laminar flow hood. Following mixing, the particles were either allowed to settle, with as much liquid (spent buffer) as possible siphoned from the container, or the particles were sonicated on a high power setting for about 30 minutes at room temperature. The particles were tested for endotoxin concentration and pH and characterized as to particle size with a Coulter N4Plus Submicron Particle Sizer. Following preparation the particles were allowed to equilibrate for approximately seven days before use.

### EXAMPLE 2

CAP Particles Impregnated by Polyethylene Glycol and Coated with Therapeutic Agent, Such as Insulin.

Particles having a surface modifying agent (2), such as polyethylene glycol (PEG), impregnated within the core calcium phosphate particle (4) and having a material (6), such as a therapeutic agent, and more particularly human insulin, at least partially coated on the surface are shown in FIG. 2C. Particles having at least a partial coating of human insulin were prepared by simultaneously injecting 5 mL of 125 mM CaCl₂ and 1 mL of 156 mM sodium citrate into a 250 mL beaker containing 100 mL of 1% polyethylene glycol (PEG),under constant stirring. Precipitate was formed following the addition of 5 mL of 125 mM Na₂HPO₄. Mixing was continued for 48 hours at room temperature. The resulting particle suspension was sonicated at maximum power for 15 minutes and stored at room temperature until ready for insulin attachment.

A therapeutic agent, in this example, human insulin at final concentration between 0.9-1.0 mg/mL (achieved by titrating the particle suspension with small volumes of insulin stock solution until the appearance of the suspension becomes milky white, resulting in a final concentration commonly around 0.95 mg/ml for most preparations, but not required) was incubated with batches of the 20 mL PEG-impregnated or incorporated particle suspension for 1 hour at room temperature by gentle mixing on a rocking platform. Finished particles were washed twice in distilled water and stored either at about 4° C. (preferably not longer than 1 month). Illustrative particles are shown schematically in FIG. 2C. Incorporating a surface modifying agent such as PEG in the particle structure results in increased loading capacity for therapeutic agent, such as insulin, measured as mg bound-insulin/100 mg particle (44±4% w/w), increased insulin per particle (12.5 U/mg particle, based on recombinant insulin unit by HPLC (high-performance liquid chromatography)=28.4 U/mg protein), and increased loading efficiency of 40.0±3.6% w/w, measured by mg bound-insulin/100 mg insulin originally added during binding.

### EXAMPLE 3

CAP-PEG-Ins (CAPI) Formulation.

Particles having both a surface modifying agent (2) and a material (6), such as a therapeutic agent impregnated within the core calcium phosphate particle (4) are shown in FIG. 3. The following materials were used as purchased to prepare the particle suspension comprising insulin and PEG incorporated in biodegradable calcium phosphate: Recombinant human insulin (Ins) (28 IU/mg) expressed in E. coli (Sigma, St. Louis, Mo.), PEG-3350 (Sigma), lyophilized bovine casein (Cas) (Sigma), calcium chloride dihydrate (Mallinckrodt, Paris, Ky.), sodium citrate dihydrate (Mallinckordt), dibasic sodium phosphate (Mallinckrodt), calcium-and magnesium-free Dulbecco's phosphate buffered saline (PBS) (Life Technologies, Grand island, N.Y.).

A stock solution of 20 mg/ml HINS was prepared in 0.01 N HCl. One volume (1 V) of insulin was diluted to 1 mg/ml using an aqueous solution of 1% (w/v) PEG and mixed thoroughly for about 1 min. Aqueous solutions of sodium citrate (0.2 V of 156 mM) and calcium chloride (1 V of 125 mM) were injected into PEG-Ins solution, simultaneously, while stirring. Solution is slightly turbid at start but it clears up instantly. Calcium phosphate formation was initiated by adding 1 V of 125 mM dibasic sodium phosphate into the reaction mixture. Mixing was continued for 40-48 hr at room temperature. The resulting particle suspension was centrifuged at about 4500× g for 15 min at 4° C. to remove any unreacted or excess components. Particles were resuspended in distilled water. Fifty mL to 100 mL aliquots of particle suspension were sonicated (550 Sonic Dismembrator, Fisher scientific) at a maximum power setting of 10 for 15-30 min in flat-bottom glass bottles. Sonicated particle suspension was centrifuged as above and the supernatant was decanted. Resulting particle pellet was either lyophilized to dryness at −50° C. under reduced pressure (25×10⁻³ mbar), or resuspended in distilled water. Final formulations were stored tightly-capped at 4° C. until further processing. Suspension formulation (without excipients or preservatives) was found very stable at 4° C. for over 2 weeks (no more than 5% insulin leakage during this period).

Measurement of Insulin Loading Capacity.

Given the fact that the only protein component in the CAP-PEG-Ins formulation is insulin, the fractions generated in the process were assayed for total insulin according to the Bradford's method using the Bio-Rad Protein Assay kit and the human insulin as the protein standard. Known amounts of lyophilized particles were solubilized in 0.01 N HCl to free encapsulated-insulin into solution. Drug loading capacity and the loading efficiency of the particles were assessed using the following equations:

Loading capacity (% w/w)=(M_{bound}/M_{particle})×100 (1)

Loading efficiency (% w/w)=(M_{bound}/M_{theoretical})×100 (2)

where M_{bound} is the amount of insulin (mg) eluted from the particles (bound-insulin), M_{particle} is the amount of particle (mg) utilized for insulin binding, and M_{theoretical} is the theoretical loading amount of insulin originally added into reaction vessel.

According to equations 1 and 2, insulin loading capacity of the formulation was 65±5% (0.65±0.05 mg insulin/mg lyophilized particle) and about 70±5% of the initially present insulin was incorporated in the final formulation.

Table 1 below shows the relative insulin loading capacities for the formulations described herein. Note that the CAPIC-1 and CAPIC-2 formulation are described in Examples 4 and 6, respectively, below.

TABLE 1
Preparation of CAP-PEG-Ins-Casein Formulation (CAPIC) for Oral Delivery

| | Initial Cas:CAPI | Final Cas:CAPI | Initial Cas:Ins | Final Cas:Ins | % Insulin¹ |
|---|---|---|---|---|---|
| Formulation | (mg/mg) | (mg/mg) | (mg/mg) | (mg/mg) | (w/w) |
| CAP-PEG-Ins | n.a. | n.a. | n.a. | n.a. | 65 |
| Particles (CAPI) | | | | | |
| CAPI-Cas-1 | 0.80 | 0.32 | 1.33 | 0.53 | 40 |
| (CAPIC-1) | | | | | |
| CAPI-Cas-2 | 1.60 | 0.96 | 2.67 | 1.60 | 30 |
| (CAPIC-2) | | | | | |

n.a. Not applicable

Note that lower % insulin capacities for CAPIC formulations in comparison to CAPI are not due to any loss of insulin during formulation but due to addition of casein around the particles and subsequent weight increase of the final formulation. In the above examples, all formulations contained approximately 15 mg total insulin initially.

### EXAMPLE 4

CAP-PEG-Insulin-casein (CAPIC-1) oral formulation.

PBS was diluted by 1:2 in distilled water and pH was adjusted to 8 using 1N HCl (½ PBS). A 1 mg/ml casein (Cas) solution was prepared by dispersing the appropriate amount of powdered bovine casein in V₂ PBS, pH 8, and mixing for about 2 hrs at room temperature. About 25 mg of CAP-PEG-Ins containing about 15 mg insulin (420 IU) was dispersed in 20 ml casein solution (20 mg casein). The mixture was rotated for about 2 hr at room temperature and incubated overnight at 4° C. by gentle stirring. The pH of the mixture at 4° C. was about 7.5. Control for the experiment involved CAP-PEG-ins particles resuspended in PBS, pH 8. Since insulin is soluble at acidic conditions (pH 2-3) and has a very low resolubility around neutral pH, no significant insulin leakage under the process conditions was anticipated. Precipitation of caseins, presumably as micelles, around CAP-PEG-Ins particles were indicated by the formation of a white-milky appearance in the suspension. Reformed casein micelles surrounding the core of CAP-PEG-Ins were collected by centrifugation and lyophilized to dryness.

Dry weight of the final product indicated that approximately 8 mg of initially present casein (40% w/w) was precipitated around CAPI (˜0.3 mg casein/mg particle) (Table 1). To test our assumption that no insulin was leaked from the formulation during the process, control CAP-PEG-Ins particles at pH 8 was assayed for insulin using the Bradford's protein assay. No insulin was detected in the supernatant fraction and 98% of the original insulin remained incorporated within the particle structure. Thus, it was estimated that CAPIC-1 contained approximately 40% insulin by weight (0.4 mg/mg or about 10 IU /mg).

### EXAMPLE 5

Stability of CAPIC-1 Oral Formulation Against Digestive Enzymes

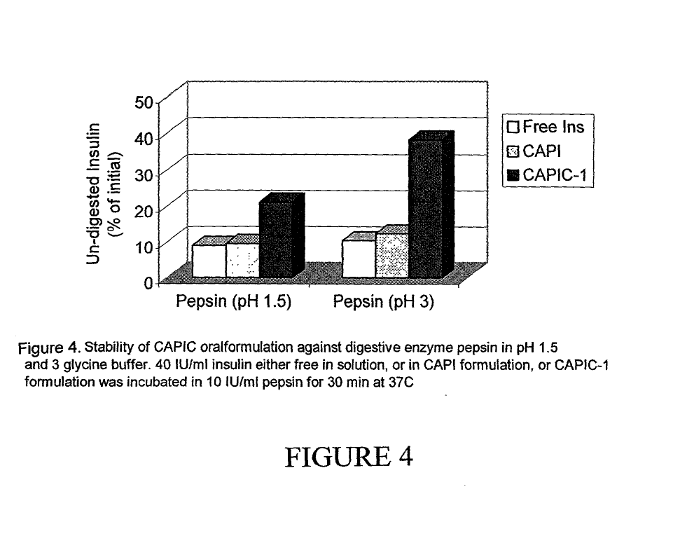

Pepsin (10 U/ml) was prepared in pH 1.5 or pH 3 glycine buffer. A 4 mg/ml CAPIC-1 dispersion (40 IU insulin/ml) was prepared in distilled water. Equal volumes of enzyme and CAPIC solutions were mixed and incubated at 37° C. for 30 min. The final suspensions contained 20 IU of insulin/milliliter of incubation medium. Forty IU/ml insulirt either free in solution, in CAPI formulation, or CAPIC-1 formulation was incubated in 10 IU/ml pepsin for 30 minutes at 37° C. In other words, free insulin, CAPI, and CAPIC-1 in distilled water were treated identically for comparison. Enzyme-treated CAPI and CAPIC-1 were collected by centrifugation and washed once with distilled water. Washed pellets were completely digested in pepsin, pH 1.5. Fractions were analyzed by a combination of the Bradford's method and ELISA for insulin using insulin as the protein standard. Results indicated that while only 10% of initially present free insulin was left undigested at pH 1.5 and pH 3, greater than 20% of insulin at pH 1.5 and about 40% of insulin at pH 3 remained undigested in CAPIC-1 formulation (See FIG. 4).

### EXAMPLE 6

CAP-PEG-Insulin-Casein-2 (CAPIC-2) Oral Formulation.

In effort to increase the enzyme resistance of CAPIC formulation, initial casein to CAP-PEG-Ins ratio (0.8:1 w:w) in the first example was increased to 1.6:1. The procedure of CAPIC- 1 synthesis was repeated with the following modifications: 1) Instead of CAPI suspension, lyophilized formulation was used; 2) Instead of 1 mg/ml casein solution in Example 1, a 2 mg/ml solution was prepared. CAPIC-2 comprising casein-coated CAPI was synthesized as in Example 4. Final formulation was prepared as a lyophilized powder as before. Dry weight determinations from multiple preparations indicated that about 60% (w/w) of original casein was precipitated (reformed) as micelles around CAP-PEG-Ins particles (˜1 mg casein/mg CAPI). CAPIC-2 formulation contained approximately 30% (w/w) insulin.

### EXAMPLE 7

Oral Administration of CAPIC-2 to Diabetic Mice.

Non-obese diabetic (NOD) female mice at 13-14 weeks of age were used to assess the in-vivo activity of CAPIC-2 as an oral delivery system. Animals were divided into 3 groups of 4-6 mice. Average body weights were determined before the treatment started. The protocol used in the study was approved by the local IACUC. Effect of oral formulation on whole blood glucose levels was the only assessment variable. A glucometer and glucose strips were used to determine pre- and post-treatment blood glucose levels.

Lyophilized CAPIC-2 was resuspended in distilled water and vortexed vigorously to obtain a homogenous suspension. Final insulin concentration was adjusted to 40 IU/ml. Similarly, 40 IU/ml aqueous solutions of unmodified (free) insulin was prepared from a stock solution of 20 mg/ml in 0.01 N HCl for subcutaneous and oral administrations as controls. The night before the treatment started, animals were transiently anaesthetized with metaphane inhalation. Fifty μl to 100 μl blood was collected from the orbital sinus and immediately dropped onto a glucose strip. Whole blood glucose level was recorded directly from the glucometer reading. Following a 30 min resting period with food and water, food was removed from cages and animals were fasted overnight (about 15 hrs) to reduce basal insulin levels. They had free access to water.

Post-fasting glucose levels were determined as before. Following a 30 min resting period, the first group of 6 mice received a single dose of 100 U/Kg body weight CAPIC in 100 μl solution directly into stomach by oral intubation. The second group of 4 mice received one single dose of 100 U/kg aqueous solution of free insulin. The third group of 6 mice received one single dose of 12.5 IU/Kg of free insulin by subcutaneous injection. Mice injected with insulin at doses higher than 12.5 IU/Kg developed immediate and sever hypoglycemia and went in hypoglycemic shock during preliminary dose-response testing (data not included). Thus, 100 IU/Kg free insulin could not be administered by subcutaneous route. Blood was drawn from treated animals every 0.5-1 hr during the first 6 hr, then 10 and 24 hr after the insulin administration. Blood glucose was measured as before.

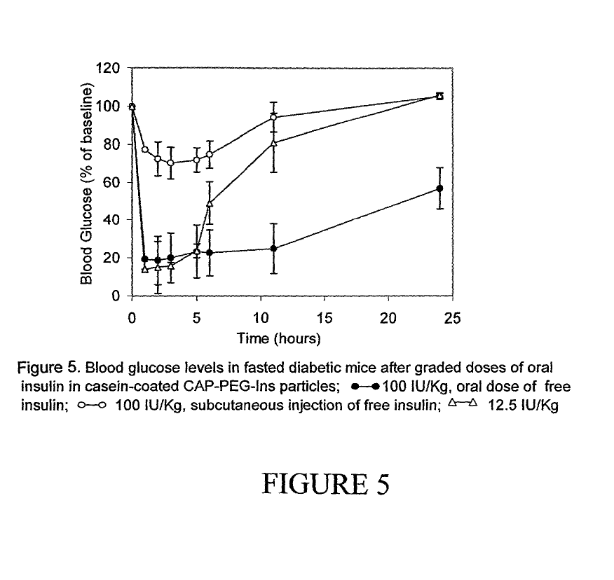

Change in blood glucose levels following the insulin administration was plotted as a percentage of post-fasting glucose (baseline) levels in FIG. 5.

Oral administration of CAPIC formulations at 12.5-25 IU/Kg insulin doses did not produce any significant reduction in blood glucose levels (results not shown).

### SUMMARY OF RESULTS

Oral administration of 100 IU/Kg of insulin as casein-coated CAP-PEG-Insulin (the CAPIC formulations) produced significant reductions in fasted-blood glucose levels 30 minutes after administration. Blood glucose levels dropped approximately 20% of initial post-fasting glucose levels (80% decrease) and remained at that level for at least 10 hours after testing. At 24 hours after testing, glucose levels remained significantly lower than the starting levels (40% of baseline). When an equal dose of unmodified insulin was given orally in solution, only about a 25% decrease in glucose levels was observed, which lasted for 5 hours after administration. Baseline glucose levels were reached within the next few hours and subsequently remained unchanged.

Glycemic affect of oral administration of CAPIC-2 formulation was also compared with that of conventional subcutaneous route. Reduction in blood glucose levels after subcutaneous injection of 12.5 IU/Kg insulin solution was almost the same order (˜80% reduction) of that demonstrated by 100 IU/Kg CAPIC-2 oral administration during the first 4 hr of testing. Glucose levels gradually increased after 4 hours, and 70% of the initial glucose level was reached 10 hours after the subcutaneous administration was recorded.

The results show that CAPIC formulations provide a therapeutic, pharmacological formulation capable of reducing blood glucose levels when administered orally. The CAPIC formulations of this invention comprise casein micelles encapsulating insulin as an integral part of a biodegradable, non-toxic microparticle preparation composed of calcium phosphate and PEG (CAPI). Calcium phosphate-based CAPI particles were used to reform casein micelles from an aqueous solution of bovine casein. As a result, CAPI, and thus insulin, was coated with a protective casein layer which facilitated the safe passage of insulin across the gastrointestinal tract to the small intestines and eventually into the blood stream.

Accordingly, casein entrapped CAP particles can be used as an oral insulin delivery system. It should be understood that the described process parameters may be modified to prepare better formulations to provide more protection for insulin in acidic media (such as in stomach) and to provide the desired bioavailability (release) in the less acidic or more basic pH conditions (such as in the small intestine).

For example, in an effort to produce a more acid-resistant oral formulation, CAPIC may be crosslinked with 4% glutaraldehye. Results indicate that glutaraldehyde-crosslinked casein entrapped CAP particles may facilitate further protection for therapeutic agents in acidic pHs (e.g. about 60% of the loaded insulin remains undigested at pH 3).

The procedures described and exemplified above can be modified by those having skill in the art to yield other embodiments of the invention. For example, the material to be dispersed throughout the particle can be co-crystallized and impregnated within the particle as described above, and the resulting particles can be coated with the same or different material, using the coating methods described above. The particle cores may also have a partial coating of one or a mixture of surface modifying agents described above to help adhere material coating the particle to the surface thereof, or to confer additional controlled-release possibilities on the drug or the active pharmaceutical component.

The present invention has been described above with respect to certain specific embodiments thereof, however it will be apparent that many modifications, variations, and equivalents thereof are also within the scope of the invention.

What is claimed is:

1. A particle, comprising:
(1) a core, comprising calcium phosphate;
(2) a therapeutic agent associated with the core; and
(3) a layer comprising casein at least partially covering the core.

2. The particle of claim 1, wherein the therapeutic agent is selected from the group consisting of insulin, Alpha-1-Antitrypsin, Human Growth Hormone (HGH); Erythropoeitin (EPO), Steroids, drugs to treat osteoporosis, blood coagulation factors, anti-cancer drugs, antibiotics, lipase, garanulocyte-colony stimulating factor (G-CSF), Beta-Blockers, anti-asthma, anti-sense oligonucleotides, therapeutic antibodies, DNase enzyme for respiratory diseases, anti-inflammatory drugs, anti-virals, anti-hypertensives, cardiotherapeutics, anti-arrythmia drugs, gene therapies; diuretics, anti-clotting chemicals, and any combination thereof.

3. The particle of claim 1, wherein the particle size ranges from about 300 nm to about 10 microns.

4. The particle of claim 1, wherein the therapeutic agent is at least partially coated on the outside of the core, at least partially encapsulated within the core, or a combination of both.

5. The particle of claim 1, further comprising a surface modifying agent at least partially coated on the outside of the core, at least partially embedded within the core, or a combination of both.

6. The particle of claim 5, wherein the surface modifying agent is selected from the group consisting of basic sugars, modified sugars, polyethylene glycol, cellobiose, oligonucleotides, carbohydrates, carbohydrate derivatives, macromolecules with carbohydrate-like components, and combinations thereof.

7. A therapeutic composition comprising the particle of claim 1 and a pharmaceutically acceptable excipient.

8. The therapeutic composition of claim 7, wherein the therapeutic agent is insulin.

9. A therapeutic composition suitable for oral delivery of insulin, comprising:
(1) a core comprising calcium phosphate;
(2) insulin and polyethylene glycol associated with the core; wherein the insulin and polyethylene glycol are at least partially encapsulated within the core;
(3) a capsule comprising casein at least partially covering the core;
wherein the capsule is combined with a pharmaceutically acceptable excipient.

10. A method of preparing one or more particles having calcium phosphate complexed with a therapeutic agent to form a particle, wherein the particle is encapsulated by casein, comprising:
(a) reacting a soluble calcium salt, a soluble phosphate salt, and the therapeutic agent to form a mixture;
(b) dispersing the mixture in a solution of casein.

11. The method of claim 10, wherein the reacting (a) further comprises:
(i) mixing the therapeutic agent with a surface modifying agent; and
(ii) reacting the soluble calcium salt and the soluble phosphate salt with the therapeutic agent and surface modifying agent to form the mixture.

12. A method for delivering a therapeutic amount of a therapeutic agent to a patient in need thereof, comprising orally delivering one or more particles of claim 1.
